(19) United States
(12) Patent Application Publication
(10) Pub. No.: US 2026/0315349 A1
(43) Pub. Date: Oct. 15, 2026

(54) STABLE UV-SHIELDING COMPOSITION

(71) Applicant: L'OREAL, PARIS (FR)

(72) Inventors: Yuan LUO, Kawasaki-shi (JP); Noemie BROMBERGER, Kawasaki-shi (JP); Jun SUZUKI, Kawasaki-shi (JP)

(21) Appl. No.: 19/491,536

(22) PCT Filed: May 14, 2024
(86) PCT No.: PCT/JP2024/080074

§ 371 Date: Dec. 9, 2025

(30) Foreign Application Priority Data
Jun. 19, 2023 (JP) 2023-100179
Jul. 28, 2023 (FR) FR2308204

(51) Int. Cl. A61K 8/37; A61K 8/06; A61K 8/25; A61K 8/29; A61K 8/34; A61K 8/40; A61K 8/44; A61K 8/49; A61Q 17/04

(52) U.S. Cl. CPC A61K 8/375; A61K 8/062; A61K 8/25; A61K 8/29; A61K 8/342; A61K 8/37; A61K 8/40; A61K 8/442; A61K 8/4966; A61Q 17/04

(57) ABSTRACT

A composition contains: (a) at least one amino acid surfactant; (b) at least one polyglyceryl fatty acid ester; (c) at least one UV filter; and (d) at least one fatty alcohol. The HLB value of the (b) polyglyceryl fatty acid ester(s) is 6 or more, preferably 7 or more, and more preferably 8 or more, and the fatty acid moiety of the (b) polyglyceryl fatty acid ester comprises carbon atoms of 16 or less, preferably 14 or less, and more preferably 12 or less. The composition is stable and can form a homogeneous film which provides good UV protection.

### TECHNICAL FIELD

The present invention relates to a composition, preferably a cosmetic or dermatological composition, which is stable, comprising at least one UV filter.

### BACKGROUND ART

Currently, one technical challenge in cosmetic fields is to provide a stable composition with high UV protection capability as well as smooth or non-sticky texture upon application.

Conventional suncare compositions with a high concentration of UV filter(s) for high UV protection performance need to have a high concentration of surfactant(s) as well to secure good stability of the compositions. However, such a high concentration of surfactant(s) tends to cause heavy or sticky texture upon application.

### DISCLOSURE OF INVENTION

A UV protection performance of a composition including UV filter(s) depends on the homogeneity of a film, including the UV filter(s), formed by the composition.

An objective of the present invention is to provide a stable composition which can provide a homogeneous film that provides good UV protection.

The above objective of the present invention can be achieved by a composition comprising:
(a) at least one amino acid surfactant;
(b) at least one polyglyceryl fatty acid ester;
(c) at least one UV filter; and
(d) at least one fatty alcohol,
wherein
the HLB value of the (b) polyglyceryl fatty acid ester(s) is 6 or more, preferably 7 or more, and more preferably 8 or more, and
the fatty acid moiety of the (b) polyglyceryl fatty acid ester comprises carbon atoms of 16 or less, preferably 14 or less, and more preferably 12 or less.

The (a) amino acid surfactant may be selected from the compounds represented by the formula (A):

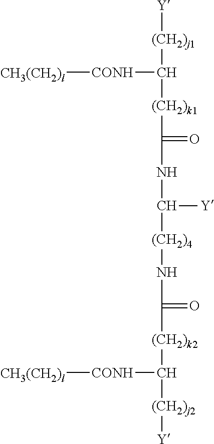

wherein
Y′ independently denotes a carboxylic acid group or an alkaline salt of a carboxylic acid group such as a sodium salt of a carboxylic acid group,
j1, k1, j2 and k2 denote integers such that (j1, k1, j2, k2)=any of (2,0,2,0), (2,0,0,2), (0,2,2,0) and (0,2,0,2), and
l denotes an integer from 6 to 16, preferably from 8 to 14, and more preferably from 10 to 12.

The (a) amino acid surfactant may be selected from the group consisting of sodium dilauramidoglutamide lysine, sodium dimyristoylglutamide lysine, and sodium distearoylglutamide lysine, and a mixture thereof.

The amount of the (a) amino acid surfactant(s) in the composition according to the present invention may range from 0.01% to 10% by weight, preferably from 0.05% to 5% by weight, and more preferably from 0.1% to 1% by weight, relative to the total weight of the composition.

The (b) polyglyceryl fatty acid ester may be selected from the group consisting of PG2 laurate, PG4 caprate, PG5 laurate, PG6 dicaprate, PG6 caprylate, PG10 laurate, and a mixture thereof. The amount of the (b) polyglyceryl fatty acid ester(s) in the composition according to the present invention may range from 0.01% to 10% by weight, preferably from 0.05% to 5% by weight, and more preferably from 0.1% to 2% by weight, relative to the total weight of the composition.

The (c) UV filter may be selected from organic UV filters, and preferably from the group consisting of butyl methoxydimenzoylmethane, bis-ethylhexyloxyphenol methoxyphenyl triazine, ethylhexyl triazone, drometrizole trisiloxane, ethylhexyl salicylate, octocrylene, homosalate, and a mixture thereof.

The amount of the (c) UV filter(s) in the composition according to the present invention may range from 1% to 30% by weight, preferably from 5% to 25% by weight, and more preferably from 10% to 20% by weight, relative to the total weight of the composition.

The (d) fatty alcohol may be selected from the group consisting of cetyl alcohol, stearyl alcohol, arachidyl alcohol, behenyl alcohol, and a mixture thereof.

The amount of the (d) fatty alcohol(s) in the composition according to the present invention may range from 0.01% to 10% by weight, preferably from 0.05% to 5% by weight, and more preferably from 0.1% to 2% by weight, relative to the total weight of the composition.

The composition according to the present invention may further comprise (e) water.

The amount of the (e) water in the composition according to the present invention may range from 40% to 70% by weight, preferably from 45% to 65% by weight, and more preferably from 50% to 60% by weight, relative to the total weight of the composition.

The composition according to the present invention may be in the form of an O/W emulsion.

The composition according to the present invention may be a cosmetic composition, preferably a cosmetic composition for a keratin substance, and more preferably a cosmetic composition for skin.

The present invention also relates to a cosmetic process for treating a keratin substance, comprising the step of applying the composition according to the present invention.

### BEST MODE FOR CARRYING OUT THE INVENTION

After diligent research, the inventors have discovered that it is possible to provide a stable composition which can provide a homogeneous film that provides good UV protection.

Thus, one aspect of the present invention is a composition comprising:
(a) at least one amino acid surfactant;
(b) at least one polyglyceryl fatty acid ester having an HLB value of 8 or more;
(c) at least one UV filter; and
(d) at least one fatty alcohol,
wherein
the HLB value of the (b) polyglyceryl fatty acid ester(s) is 6 or more, preferably 7 or more, and more preferably 8 or more, and
the fatty acid moiety of the (b) polyglyceryl fatty acid ester comprises carbon atoms of 16 or less, preferably 14 or less, and more preferably 12 or less.

The composition according to the present invention is UV shielding or is capable of shielding UV rays, because it includes (c) at least one UV filter.

The composition according to the present invention can form a homogeneous or uniform film including the (c) UV filter(s) that can provide a homogeneous or uniform distribution of the (c) UV filter(s), which can provide good UV protection.

The composition according to the present invention is stable. The term “stable” here means that the aspect of a composition does not change, for example, as a result of aggregation or phase separation. It is preferable that the composition according to the present invention is stable over a long time of period.

Also, the composition according to the present invention can provide good texture such as a smooth or non-sticky feeling to touch during application. In other words, the composition according to the present invention is not sticky upon application onto a keratin substance such as skin. The term “sticky” here means a property which provides a tacky feeling to the skin.

In summary, the composition according to the present invention is stable and can form a homogeneous or uniform film which can provide good UV protection. In addition, the composition according to the present invention can provide good texture such as a smooth or non-sticky feeling to touch during application.

Thus, the composition according to the present invention is suitable for cosmetic products, in particular suncare cosmetic products such as a skin care cream.

Hereinafter, the composition according to the present invention will be explained in a more detailed manner.

### [Amino Acid Surfactant]

The composition according to the present invention comprises (a) at least one amino acid surfactant. A single type of amino acid surfactant may be used, but two or more different types of amino acid surfactant may be used in combination.

The (a) amino acid surfactant is an anionic surfactant based on an amino acid or derivatives thereof. Typically, the (a) amino acid surfactant is an anionic surfactant comprising at least one amino moiety and at least one carboxylic acid moiety which is in the form of a carboxylate. The (a) amino acid surfactant may have two or more amino moieties and/or two or more carboxylic acid moieties which are in the form of carboxylates.

The (a) amino acid surfactant may preferably be selected from amino acid derivatives. The amino acid derivative may more preferably be selected from salts of amino acids and N-acylated amino acids, for example alkali metal salts and alkali earth metal salts of amino acids and N-acylated amino acids, such as sodium salts, potassium salts, magnesium salts, and calcium salts of amino acids and N-acylated amino acids.

The acyl group which forms the N-acyl moiety of the amino acid derivatives may be a C₁-C₃₀ acyl group, preferably a C₆-C₂₈ acyl group, and more preferably a C₁₂-C₂₄ acyl group.

The (a) amino acid surfactant may even more preferably be selected from the group consisting of glutamates, N-acylated glutamates, aspartates, N-acylated aspartates, and salts thereof.

Examples of the (a) amino acid surfactant include, but are not limited to:
sarcosinates, such as N-acyl sarcosinates (salts), e.g., sodium lauroyl sarcosinate, sold under the name Nikkol Sarcosinate™ LN by Nikko Chemicals or sold under the name Pureact™ LSR by Innospec Performance Chemicals Europe Limited; sodium myristoyl sarcosinate, sold under the name Nikkol Sarcosinate™ MN by Nikko Chemicals; and sodium palmitoyl sarcosinate, sold under the name Nikkol Sarcosinate™ PN by Nikko Chemicals;
alaninates, such as N-acyl alaninates (salts), e.g., sodium N-lauroyl-N-methylamidopropionate, sold under the name Sodium Nikkol Alaninate™ LN 30 by Nikko Chemicals or sold under the name Alanone® ALE by Kawaken; sodium cocoyl alaninates, sold under the name Amilite® ACS-12 by Ajinomoto; and triethanolamine N-lauroyl-N-methylalanine, sold under the name Alanone® ALTA by Kawaken;
glutamates, such as N-acyl glutamates (salts), e.g., sodium stearoyl glutamate, sold under the name Eumulgin® SR by BASF Japan; sodium lauroyl glutamate, sold under the name Plantapon® Amino SLG-P by BASF Japan; triethanolamine monococoyl glutamate, sold under the name Amisoft® CT-12 by Ajinomoto; triethanolamine lauroyl glutamate, sold under the name Amisoft® LT-12 by Ajinomoto; and disodium stearoyl glutamate, sold under the name Amisoft® HS-21P by Ajinomoto;
aspartates, such as N-acyl aspartates (salts), e.g., disodium N-lauroyl aspartate, sold under the name AminoFoamer® FLMS-P1 by Asahi Kasei; and a mixture of triethanolamine N-lauroyl aspartate and triethanolamine N-myristoyl aspartate, sold under the name AminoFoamer® FCMT-L by Asahi Kasei; and
glycinates, such as N-acyl glycinates (salts), e.g., sodium N-cocoyl glycinate, sold under the name Amilite® GCS-12K; and potassium N-cocoyl glycinate, sold under the name Amilite® GCK-12K by Ajinomoto.

It is preferable that the (a) amino acid surfactant be selected from amino acid gemini surfactants which are gemini surfactants based on amino acids.

Gemini surfactants or dimeric surfactants are well known. For a detailed description of the various chemical structures and their physicochemical properties, reference may be made to the following publications:
Milton J. Rosen, Gemini Surfactants, Properties of surfactant molecules with two hydrophilic groups and two hydrophobic groups, Cosmetics & Toiletries magazine, Vol. 113, December 1998, pp. 49-55; and
Milton J. Rosen, Recent Developments in Gemini Surfactants, Allured's Cosmetics & Toiletries magazine, July 2001, Vol. 116, No. 7, pp. 67-70.

The amino acid gemini surfactant is anionic and comprises a plurality of hydrophobic moieties and a plurality of hydrophilic moieties.

The amino acid gemini surfactant may be selected from compounds with two or more fatty amide groups (hereafter, it may be referred to as “amide compound”). The amide compound may be represented by the following formula (A):

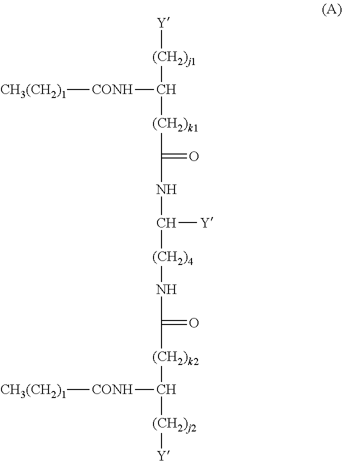

wherein
Y′ independently denotes a carboxylic acid group or an alkaline salt of a carboxylic acid group such as a sodium salt of a carboxylic acid group,
j1, k1, j2 and k2 denote an integer such that (j1, k1, j2, k2)=any of (2,0,2,0), (2,0,0,2), (0,2,2,0) and (0,2,0,2), and
l denotes an integer from 6 to 16, preferably 8 to 14, and more preferably 10 to 12.

Preferably, in formula (A), l denotes an integer from 8 to 12, j1=j2=0, and k1=k2=2.

Most preferably, in formula (A), Y′ is —COONa, j1=j2=0, k1=k2=2; and l=10.

As the above amide compound represented by formula (A), mention may be made of sodium dilauramidoglutamide lysine, sodium dimyristoylglutamide lysine, and sodium distearoylglutamide lysine. Sodium dilauramidoglutamide lysine is particularly preferable. Sodium dilauramidoglutamide lysine is marketed as PELLICER L-30 by Asahi Chemicals as an aqueous solution at a concentration of 29% by weight relative to the total weight of the aqueous solution or PELLICER LB-30G by Asahi Chemicals as an aqueous solution at a concentration of 27.3% by weight relative to the total weight of the aqueous solution.

The above amide compound represented by formula (A) can be prepared by, for example, reacting a long chain N-acyl acidic amino acid anhydride with a basic amino acid, such as lysine, in water and/or a mixed solvent of water and organic solvent(s), or in inert organic solvent(s) such as tetrahydrofuran, benzene, toluene, xylene tetrachloromethane, chloroform, acetone or the like, or without any solvent, at −5° C. to 200° C., preferably 5° C. to 100° C., and more preferably 0° C. to 60° C.

Thus, the amino acid gemini surfactant may be a N-acylglutamate/amino acid derivative, preferably a dipeptide of two N-acylglutamates and a basic amino acid such as lysine.

The amount of the (a) amino acid surfactant(s) in the composition according to the present invention may be 0.01% by weight or more, preferably 0.05% by weight or more, and more preferably 0.1% by weight or more, relative to the total weight of the composition.

On the other hand, the amount of the (a) amino acid surfactant(s) in the composition according to the present invention may be 10% by weight or less, preferably 5% by weight or less, and more preferably 1% by weight or less, relative to the total weight of the composition.

Thus, the amount of the (a) amino acid surfactant(s) in the composition according to the present invention may range from 0.01% to 10% by weight, preferably from 0.05% to 5% by weight, and more preferably from 0.1% to 1% by weight relative to the total weight of the composition.

### [Polyglyceryl Fatty Acid Ester]

The composition according to the present invention comprises (b) at least one polyglyceryl fatty acid ester with specific conditions. A single type of such a polyglyceryl fatty acid ester may be used, but two or more different types of such polyglyceryl fatty acid esters may be used in combination.

The (b) polyglyceryl fatty acid ester used for the present invention has an HLB value of 6 or more, preferably 7 or more, and more preferably 8 or more.

The (b) polyglyceryl fatty acid ester used for the present invention may have an HLB value of 18 or less, preferably 17 or less, and more preferably 16 or less.

Thus, the (b) polyglyceryl fatty acid ester used for the present invention may have an HLB value of from 6 to 18, preferably from 7 to 17, and more preferably from 8 to 16.

If two or more (b) polyglyceryl fatty acid esters are used, the HLB value is determined by the weighted average of the HLB values of all the (b) polyglyceryl fatty acid esters.

The fatty acid moiety of the (b) polyglyceryl fatty acid ester used for the present invention comprises carbon atoms of 16 or less, preferably 14 or less, and more preferably 12 or less. If the (b) polyglyceryl fatty acid ester has two or more fatty acid moieties, each of the fatty acid moieties comprises carbon atoms of 16 or less, preferably 14 or less, and more preferably 12 or less.

The fatty acid moiety of the (b) polyglyceryl fatty acid ester used for the present invention may comprise carbon atoms of 4 or more, preferably 5 or more, and more preferably 6 or more. If the (b) polyglyceryl fatty acid ester has two or more fatty acid moieties, each of the fatty acid moieties may comprise carbon atoms of 4 or more, preferably 5 or more, and more preferably 6 or more.

Thus, the fatty acid moiety of the (b) polyglyceryl fatty acid ester used for the present invention may comprise from 4 to 16 carbon atoms, preferably from 5 to 14 carbon atoms, and more preferably from 6 to 12 carbon atoms. If the (b) polyglyceryl fatty acid ester has two or more fatty acid moieties, each of the fatty acid moieties may comprise from 4 to 16 carbon atoms, preferably from 5 to 14 carbon atoms, and more preferably from 6 to 12 carbon atoms.

The above (b) polyglyceryl fatty acid ester used for the present invention (hereafter, referred to as simply (b) polyglyceryl fatty acid ester) can function as a surfactant, in particular a nonionic surfactant.

The (b) polyglyceryl fatty acid ester may be chosen from mono, di, tri and more esters of saturated or unsaturated fatty acid(s).

The fatty acid for the fatty acid moiety or fatty acid moieties of the (b) polyglyceryl fatty acid ester may be saturated or unsaturated, and may be selected from the group consisting of caprylic acid, capric acid, lauric acid, myristic acid, preferably selected from the group consisting of caprylic acid, capric acid, lauric acid, and myristic acid, and more preferably selected from the group consisting of caprylic acid, capric acid, and lauric acid.

It is preferable that the (b) polyglyceryl fatty acid ester comprise from 2 to 14 glycerol units, preferably from 2 to 12 glycerol units, and more preferably from 2 to 10 glycerol units.

The (b) polyglyceryl fatty acid ester(s) may be selected from the group consisting of PG2 sesquicaprylate (HLB: about 8), PG2 caprate (BLB: 9.5), PG2 laurate (HLB: 8.5), PG3 caprate (HLB: about 14), PG4 caprylate (HLB: 14), PG4 caprate (HLB: 14), PG4 laurate (HLB: 10.3), PG5 myristate (HLB: 15.4), PG5 laurate (HLB: 11), PG6 caprate (HLB: 13.1), PG6 dicaprate (HLB: 10), PG6 caprylate (HLB: 15), PG6 laurate (ILB: 14.1), PG10 laurate (HLB: 16), PG10 myristate (HLB: 14.9), PG10 trilaurate (HLB: 10.4), PG10 cocoate (HLB: 16), PG10 dicocoate (HLB: 12), PG10 tricocoate (HLB: 9), and mixtures thereof. The “PG” is an abbreviation to mean polyglyceryl.

It may be preferable that the (b) polyglyceryl fatty acid ester(s) may also be selected from the group consisting of PG2 laurate (HLB: 8.5), PG4 caprate (HLB: 14), PG5 laurate (HLB: 11), PG6 dicaprate (HLB: 10), PG6 caprylate (HLB: 15), PG10 laurate (HLB: 16), and mixtures thereof.

The amount of the (b) polyglyceryl fatty acid ester(s) in the composition according to the present invention may be 0.01% by weight or more, preferably 0.05% by weight or more, and more preferably 0.1% by weight or more, relative to the total weight of the composition.

On the other hand, the amount of the (b) polyglyceryl fatty acid ester(s) in the composition according to the present invention may be 10% by weight or less, preferably 5% by weight or less, and more preferably 2% by weight or less, relative to the total weight of the composition.

The amount of the (b) polyglyceryl fatty acid ester(s) in the composition according to the present invention may range from 0.01% to 10% by weight, preferably from 0.05% to 5% by weight, more preferably from 0.1% to 2% by weight, relative to the total weight of the composition.

### [UV Filter]

The composition according to the present invention comprises (c) at least one UV filter. Two or more different types of (c) UV filters may be used in combination. Thus, a single type of (c) UV filter or a combination of different types of (c) UV filters may be used.

There is no limitation to the type of the (c) UV filter. The (c) UV filter can be selected from inorganic UV filters, organic UV filters, and mixtures thereof. It is preferable that the (c) UV filter be selected from organic UV filters.

The amount of the (c) UV filter(s) in the composition according to the present invention may be 1% by weight or more, preferably 5% by weight or more, and more preferably 10% by weight or more, relative to the total weight of the composition.

On the other hand, the amount of the (c) UV filter(s) in the composition according to the present invention may be 30% by weight or less, preferably 25% by weight or less, and more preferably 20% by weight or less, relative to the total weight of the composition.

The amount of the (c) UV filter(s) in the composition according to the present invention may range from 1% to 30% by weight, preferably from 5% to 25% by weight, more preferably from 10% to 20% by weight, relative to the total weight of the composition.

### (Inorganic UV Filter)

The (c) UV filter may be selected from inorganic UV filters. If two or more inorganic UV filters are used, they may be the same or different.

The inorganic UV filter used for the present invention may be active in the UV-A and/or UV-B region. The inorganic UV filter may be hydrophilic and/or lipophilic. The inorganic UV filter is preferably insoluble in solvents such as water and ethanol commonly used in cosmetics.

It is preferable that the inorganic UV filter be in the form of a fine particle such that the mean (primary) particle diameter thereof ranges from 1 nm to 50 m, preferably 5 nm to 500 nm, and more preferably 10 nm to 200 nm. The mean (primary) particle size or mean (primary) particle diameter here is an arithmetic mean diameter.

The inorganic UV filter can be selected from the group consisting of metal oxides which may or may not be coated, and mixtures thereof.

Preferably, the inorganic UV filter is selected from pigments (mean size of the primary particles: generally from 5 nm to 50 nm, preferably from 10 nm to 50 nm) formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se. Preferably, the inorganic UV filter is selected from titanium oxide, zinc oxide, and more preferably titanium oxide.

The inorganic UV filter may or may not be coated. The inorganic UV filter may have at least one coating. The coating may comprise at least one compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes such as beeswax, (meth)acrylic polymers, organic UV filters, and (per)fluoro compounds.

It is preferable for the coating to include at least one organic UV filter. As the organic UV filter in the coating, a dibenzoylmethane derivative such as butyl methoxydibenzoylmethane (Avobenzone) and 2,2′-Methylenebis[6-(2H-Benzotriazol-2-yl)-4-(1,1,3,3-Tetramethyl-Butyl) Phenol](Methylene Bis-Benzotriazolyl Tetramethylbutylphenol) marketed as “TINOSORB M” by BASF may be preferable.

In a known manner, the silicones in the coating(s) may be organosilicon polymers or oligomers comprising a linear or cyclic and branched or cross-linked structure, of variable molecular weight, obtained by polymerization and/or polycondensation of suitable functional silanes and essentially composed of repeated main units in which the silicon atoms are connected to one another via oxygen atoms (siloxane bond), optionally substituted hydrocarbon radicals being connected directly to said silicon atoms via a carbon atom.

The term “silicones” also encompasses silanes necessary for their preparation, in particular alkylsilanes.

The silicones used for the coating(s) can preferably be selected from the group consisting of alkylsilanes, polydialkylsiloxanes, and polyalkylhydrosiloxanes. More preferably still, the silicones are selected from the group consisting of octyltrimethylsilane, polydimethylsiloxanes, and polymethylhydrosiloxanes.

Of course, the inorganic UV filters made of metal oxides may, before their treatment with silicones, have been treated with other surfacing agents, in particular with cerium oxide, alumina, silica, aluminum compounds, silicon compounds, or their mixtures.

The coated inorganic UV filter may have been prepared by subjecting the inorganic UV filter to one or more surface treatments of a chemical, electronic, mechanochemical, and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filters may be titanium oxides coated:
with silica, such as the product “Sunveil” from Ikeda, and “Sunsil TIN 50” from Sunjin Chemical;
with silica and with iron oxide, such as the product “Sunveil F” from Ikeda;
with silica and with alumina, such as the products “Microtitanium Dioxide MT 500 SA” from Tayca, “Tioveil” from Tioxide, and “Mirasun TiW 60” from Rhodia;
with alumina, such as the products “Tipaque TTO-55 (B)” and “Tipaque TTO-55 (A)” from Ishihara, and “UVT 14/4” from Kemira;
with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniquema, and the product “Eusolex T-AVO” from Merck;
with alumina and with aluminum laurate, such as the product “Microtitanium Dioxide MT 100 S” from Tayca;
with iron oxide and with iron stearate, such as the product “Microtitanium Dioxide MT 100 F” from Tayca;
with zinc oxide and with zinc stearate, such as the product “BR351” from Tayca;
with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca;
with silica, with alumina and with aluminum stearate and treated with a silicone, such as the product “STT-30-DS” from Titan Kogyo;
with silica and treated with a silicone, such as the product “UV-Titan X 195” from Kemira;
with alumina and treated with a silicone, such as the products “Tipaque TTO-55 (S)” from Ishihara or “UV Titan M 262” from Kemira;
with triethanolamine, such as the product “STT-65-S” from Titan Kogyo;
with stearic acid, such as the product “Tipaque TTO-55 (C)” from Ishihara; or
with sodium hexametaphosphate, such as the product “Microtitanium Dioxide MT 150 W” from Tayca.

Other titanium oxide pigments treated with a silicone are preferably TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO₂Si₃” by Cardre, and anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

Preferably, the following coated TiO₂ can be used as the coated inorganic UV filter:
Stearic acid (and) Aluminum Hydroxide (and) TiO₂, such as the product “MT-100 TV” from Tayca, with a mean primary particle diameter of 15 nm;
Dimethicone (and) Stearic Acid (and) Aluminum Hydroxide (and) TiO₂, such as the product “SA-TTO-S4” from Miyoshi Kasei, with a mean primary particle diameter of 15 nm; Silica (and) TiO₂, such as the product “MT-100 WP” from Tayca, with a mean primary particle diameter of 15 nm;
Dimethicone (and) Silica (and) Aluminum Hydroxide (and) TiO₂, such as the product “MT-Y02” and “MT-Y-110 M3S” from Tayca, with a mean primary particle diameter of 10 nm; Dimethicone (and) Aluminum Hydroxide (and) TiO₂, such as the product “SA-TTO-S3” from Miyoshi Kasei, with a mean primary particle diameter of 15 nm;
Dimethicone (and) Alumina (and) TiO₂, such as the product “UV TITAN M170” from Sachtleben, with a mean primary particle diameter of 15 nm; and
Silica (and) Aluminum Hydroxide (and) Alginic Acid (and) TiO₂, such as the product “MT-100 AQ” from Tayca, with a mean primary particle diameter of 15 nm.

In terms of UV filtering ability, TiO₂ coated with at least one organic UV filter is more preferable. For example, Avobenzone (and) Stearic Acid (and) Aluminum Hydroxide (and) TiO₂, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxide pigments are, for example:
those marketed under the trademark “Z-cote” by Sunsmart;
those marketed under the trademark “Nanox” by Elementis; and those marketed under the trademark “Nanogard WCD 2025” by Nanophase Technologies.

The coated zinc oxide pigments are, for example:
those marketed under the trademark “Oxide Zinc CS-5” by Toshiba (ZnO coated with polymethylhydrosiloxane);
those marketed under the trademark “Nanogard Zinc Oxide FN” by Nanophase Technologies (as a 40% dispersion in Finsolv TN, C₁₂-C₁₅ alkyl benzoate);
those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane);
those marketed under the trademark “NFD Ultrafme ZnO” by Daikin (ZnO coated with phosphate of perfluoroalkyl and a copolymer based on perfluoroalkylethyl as a dispersion in cyclopentasiloxane);
those marketed under the trademark “SPD-Z1” by Shin-Etsu (ZnO coated with a silicone-grafted acrylic polymer dispersed in cyclodimethylsiloxane);
those marketed under the trademark “Escalol Z100” by ISP (alumina-treated ZnO dispersed in an ethylhexyl methoxycinnamate/PVP-hexadecene copolymer/methicone mixture);
those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C₁₂-C₁₅ alkyl benzoate with hydroxystearic acid polycondensate).

The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Mention may also be made of mixtures of metal oxides, in particular of titanium dioxide and of cerium dioxide, including a mixture of equal weights of titanium dioxide coated with silica and of cerium dioxide coated with silica marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

Coated inorganic UV filters are preferable, because the UV filtering effects of the inorganic UV filters can be enhanced. In addition, the coating(s) may help uniformly or homogeneously disperse the UV filters in the composition according to the present invention.

### (Organic UV Filter)

The (c) UV filter may be selected from organic UV filters. If two or more organic UV filters are used, they may be the same or different.

The organic UV filter can be hydrophobic or water-insoluble. The organic UV filter can function as an oily ingredient. Thus, the organic UV filter can form discontinuous or inner phases of the composition according to the present invention, if the composition according to the present invention is in the form of, for example, an O/W emulsion.

The organic UV filter may be active in the UV-A and/or UV-B region. The organic UV filter may be lipophilic or oil-soluble.

The organic UV filter may be solid or liquid. The terms “solid” and “liquid” mean a substance with no fluidity and a substance with fluidity, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from α-alkylstyrene; 4,4-diarylbutadiene compounds; guaiazulene and derivatives thereof; rutin and derivatives thereof; and mixtures thereof.

Mention may be made, as examples of the organic UV filter(s), of those denoted below under their INCI names, and mixtures thereof.
Anthranilic compounds: Menthyl anthranilate, marketed under the trademark “Neo Heliopan MA” by Haarmann and Reimer.
Dibenzoylmethane compounds: Butyl methoxydibenzoylmethane, marketed in particular under the trademark “Parsol 1789” by Hoffmann-La Roche; and isopropyl dibenzoylmethane.
Cinnamic compounds: Ethylhexyl methoxycinnamate, marketed in particular under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate.
Salicylic compounds: Homosalate (homomenthyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer.
Camphor compounds, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; and polyacrylamidomethyl benzylidene camphor, manufactured under the trademark “Mexoryl SW” by Chimex.
Benzophenone compounds: Benzophenone-1 (2,4-dihydroxybenzophenone), marketed under the trademark “Uvinul 400” by BASF; benzophenone-2 (Tetrahydroxybenzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norquay; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF;
benzophenone-12, and n-hexyl 2-(4-diethylamino-2-hydroxybenzoyl)benzoate (UVINUL A+by BASF).
β,β-Diphenylacrylate compounds: Octocrylene, marketed in particular under the trademark “Uvinul N539” by BASF; and Etocrylene, marketed in particular under the trademark “Uvinul N35” by BASF.
Triazine compounds: Diethylhexyl butamido triazone, marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine marketed under the trademark «TINOSORB S» by CIBA GEIGY, and ethylhexyl triazone marketed under the trademark «UVINUL T150» by BASF.
Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975.
Benzalmalonate compounds: Dineopentyl 4′-methoxybenzalmalonate, and polyorganosiloxane comprising benzalmalonate functional groups, such as polysilicone-15, marketed under the trademark “Parsol SLX” by Hoffmann-LaRoche.
Benzimidazole compounds, in particular, phenylbenzimidazole derivatives.
Imidazoline compounds: Ethylhexyl dimethoxybenzylidene dioxoimidazoline propionate.
Bis-benzoazolyl compounds: The derivatives as described in EP-669,323 and U.S. Pat. No. 2,463,264.
Para-aminobenzoic acid compounds: PABA (p-aminobenzoic acid), ethyl PABA, Ethyl dihydroxypropyl PABA, pentyl dimethyl PABA, ethylhexyl dimethyl PABA, marketed in particular under the trademark “Escalol 507” by ISP, glyceryl PABA, and PEG-25 PABA, marketed under the trademark “Uvinul P25” by BASF.
Methylene bis-(hydroxyphenylbenzotriazol) compounds, such as 2,2′-methylenebis[6-(2H-benzotriazol-2-yl)-4-methyl-phenol]marketed in the solid form under the trademark “Mixxim BB/200” by Fairmount Chemical, 2,2′-methylenebis[6-(2H-benzotriazol-2-yl)-4-(1,1,3,3-tetramethylbutyl)phenol]marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184 and EP-893,119, and

Drometrizole trisiloxane, marketed under the trademark “Silatrizole” by Rhodia Chimie or “Mexoryl XL” by L'Oreal, as represented below.

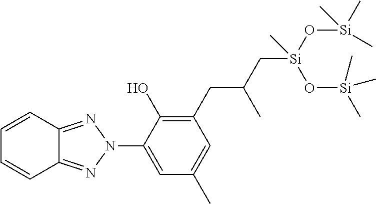 [SMILES: Cc1cc(CC(C)C[Si](C)(O[Si](C)(C)C)O[Si](C)(C)C)c(O)c(-n2nc3ccccc3n2)c1]

Benzoxazole compounds: 2,4-bis[5-1(dimethylpropyl)benzoxazol-2-yl-(4-phenyl)imino]-6-(2-ethylhexyl)imino-1,3,5-triazine, marketed under the trademark Uvasorb K2A by Sigma 3V.
Screening polymers and screening silicones: The silicones described in WO 93/04665.
Dimers derived from α-alkylstyrene: The dimers described in DE-19855649.
4,4-Diarylbutadiene compounds: 1,1-dicarboxy(2,2′-dimethylpropyl)-4,4-diphenylbutadiene.

It is preferable that the organic UV filter be selected from the group consisting of: butyl methoxydibenzoylmethane, ethylhexyl methoxycinnamate, homosalate, ethylhexyl salicylate, octocrylene, benzophenone-3, benzophenone-4, benzophenone-5, n-hexyl 2-(4-diethylamino-2-hydroxybenzoyl)benzoate, 1,1′-(1,4-piperazinediyl)bis[1-[2-[4-(diethylamino)-2-hydroxybenzoyl]phenyl]-methanone 4-methylbenzylidene camphor, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalnalonate, 1,1-dicarboxy(2,2′-dimethylpropyl)-4,4-diphenylbutadiene, 2,4-bis[5-1 (dimethylpropyl)benzoxazol-2-yl-(4-phenyl)imino]-6-(2-ethylhexyl)imino-1,3,5-triazine, camphor benzylkonium methosulfate and mixtures thereof.

It is more preferable that the organic UV filter be selected from the group consisting of butyl methoxydimenzoylmethane, bis-ethylhexyloxyphenol methoxyphenyl triazine, ethylhexyl triazone, drometrizole trisiloxane, ethylhexyl salicylate, octocrylene, homosalate, and a mixture thereof.

### [Fatty Alcohol]

The composition according to the present invention comprises (d) at least one fatty alcohol. Two or more different types of (d) fatty alcohols may be used in combination. Thus, a single type of (d) fatty alcohol or a combination of different types of (b) fatty alcohols may be used.

The term “fatty” here means the inclusion of a relatively large number of carbon atoms. Thus, alcohols which have 6 or more, preferably 8 or more, and more preferably 10 or more carbon atoms are encompassed within the scope of fatty alcohols. The fatty alcohols may be saturated or unsaturated. The fatty alcohol may be linear or branched. Two or more fatty alcohols may be used in combination.

The (d) fatty alcohol may have the structure R—OH wherein R is chosen from saturated and unsaturated, linear and branched radicals containing from 6 to 40 carbon atoms, for example from 8 to 30 carbon atoms. In at least one embodiment, R is chosen from C₁₂-C₂₄ alkyl and C₁₂-C₂₄ alkenyl groups. R may be or may not be substituted with at least one hydroxyl group.

Non-limiting examples of the (d) fatty alcohols that may be mentioned include lauryl alcohol, cetyl alcohol, stearyl alcohol, oleyl alcohol, behenyl alcohol, linoleyl alcohol, undecylenyl alcohol, palmitoleyl alcohol, arachidyl alcohol, arachidonyl alcohol, erucyl alcohol, cetearyl alcohol, and mixtures thereof.

Examples of suitable fatty alcohols include, but are not limited to, cetyl alcohol, cetearyl alcohol, stearyl alcohol, behenyl alcohol, arachidyl alcohol, and mixtures thereof.

The (d) fatty alcohol may represent a mixture of fatty alcohols, which means that several species of fatty alcohol may coexist, in the form of a mixture, in a commercial product.

According to at least one embodiment, the (d) fatty alcohol used in the composition according to the present invention is chosen from a mixture of cetyl alcohol and stearyl alcohol (cetearyl alcohol), and/or a mixture of arachidyl alcohol and behenyl alcohol.

It may be preferable that the (d) fatty alcohol be selected from the group consisting of cetyl alcohol, stearyl alcohol, arachidyl alcohol, behenyl alcohol, and a mixture thereof. The amount of the (d) fatty alcohol in the composition according to the present invention may be 0.01% by weight or more, preferably 0.05% by weight or more, and more preferably 0.1% by weight or more, relative to the total weight of the composition.

On the other hand, the amount of the (d) fatty alcohol in the composition according to the present invention may be 10% by weight or less, preferably 5% by weight or less, and more preferably 2% by weight or less, relative to the total weight of the composition.

The amount of the (d) fatty alcohol in the composition according to the present invention may be from 0.01% to 10% by weight, preferably from 0.05% to 5% by weight, and more preferably from 0.1% to 2% by weight, relative to the total weight of the composition.

### [Water]

The composition according to the present invention may comprise (e) water.

The (e) water, if present, can form an aqueous phase of the composition according to the present invention.

If the composition according to the present invention is in the form of an O/W emulsion, the (e) water, if present, in the composition according to the present invention can form a continuous aqueous phases in the O/W emulsion.

The amount of the (e) water in the composition according to the present invention may be 40% by weight or more, preferably 45% by weight or more, and more preferably 50% by weight or more, relative to the total weight of the composition.

On the other hand, the amount of the (e) water in the composition according to the present invention may be 70% by weight or less, preferably 65% by weight or less, and more preferably 60% by weight or less, relative to the total weight of the composition.

The amount of (e) water in the composition according to the present invention may range from 40% to 70% by weight, preferably from 45% to 65% by weight, more preferably from 50% to 60% by weight, relative to the total weight of the composition.

### [Oil]

The composition according to the present invention may comprise (f) at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

According to the present invention, the (f) oil is different from the (d) fatty alcohol.

The (f) oil may be a non-polar oil such as a hydrocarbon oil, a silicone oil, or the like; a polar oil such as a plant or animal oil and an ester oil or an ether oil; or a mixture thereof.

The (f) oil may be selected from the group consisting of oils of plant or animal origin, synthetic oils, silicone oils, and hydrocarbon oils.

In one embodiment, the plant oil may be selected from plant-extracted oils, plant-extracted butters, and mixtures thereof.

Among the plant-extracted oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

Among the plant-extracted butters, mention may be made of, for example, shea butter, Nilotica shea butter (Butyrospermum parkii), galam butter (Butyrospermum parkii), Borneo butter or fat or tengkawang tallow (Shorea stenoptera), shorea butter, illipe butter, madhuca butter or (Bassia) Madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya (kpangnan) butter (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter (Theobroma cacao), and sunflower butter.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), glyceryl tri(caprate/caprylate/linolenate) and glyceryl tri(caprate/caprylate/succinate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of the formula:

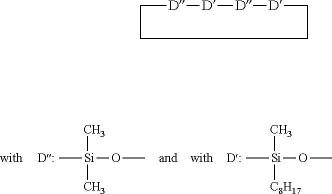

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

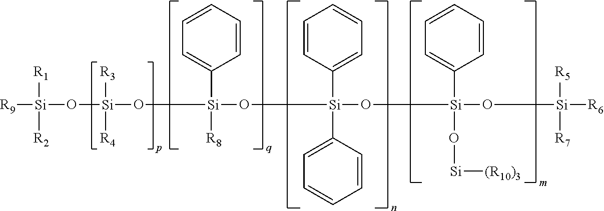

in which
R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and
m, n, p, and q are, independently of each other, integers of 0 to 900 inclusive, preferably 0 to 500 inclusive, and more preferably 0 to 100 inclusive, with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

It may be preferable that the (f) oil comprises no silicone oil.

Hydrocarbon oils may be chosen from:
linear or branched, optionally cyclic, C₅-C₁₉ lower alkanes. Examples that may be mentioned include hexane, undecane, dodecane, tridecane, and isoparaffins, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

It is preferable that the (f) oil be selected from hydrocarbon oils such as C₁₅-19 alkane, plant oils, ester oils, and mixtures thereof.

The amount of the (f) oil(s) in the composition according to the present invention may be 0.01% by weight or more, preferably 0.1% by weight or more, and more preferably 1% by weight or more, relative to the total weight of the composition.

On the other hand, the amount of the (f) oil(s) in the composition according to the present invention may be 25% by weight or less, preferably 20% by weight or less, and more preferably 15% by weight or less, relative to the total weight of the composition.

The amount of the (f) oil(s) in the composition according to the present invention may be from 0.01% to 25% by weight, preferably from 0.1% to 20% by weight, and more preferably from 1% to 15% by weight, relative to the total weight of the composition.

The (f) oil(s), if present, can form a fatty phase of the composition according to the present invention.

If the composition according to the present invention is in the form of an O/W emulsion, the (f) oil, if present, in the composition according to the present invention can form, with the (c) UV filter, if the (c) UV filter is selected from organic UV filters, dispersed fatty phases in the O/W emulsion.

### [Optional Ingredients]

The composition according to the present invention may comprise, in addition to the aforementioned ingredients, optional ingredient(s) typically employed in cosmetics, specifically, hydrophilic or lipophilic thickeners, derived from, for example, synthetic polymers; volatile or non-volatile organic solvents other than the (f) oil; anionic polymers; cationic polymers; amphoteric polymers; nonionic polymers; silicones and silicone derivatives other than the (f) oil; natural extracts derived from animals or vegetables other than the (f) oil; and the like, within a range which does not impair the effects of the present invention.

The composition according to the present invention may comprise the above optional ingredient(s) in an amount of from 0.01% to 30% by weight, preferably from 0.05% to 20% by weight, and more preferably from 0.1% to 10% by weight, relative to the total weight of the composition.

### (Preparation)

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

### [Form]

The form of the composition according to the present invention is not particularly limited, and may take various forms such as a solution and an emulsion.

It is preferable that the composition according to the present invention be in the form of an emulsion. It is more preferable that the composition according to the present invention be in the form of an O/W emulsion, which comprises fatty phases dispersed in a continuous aqueous phase. The dispersed fatty phases can be oil droplets in the aqueous phase.

The O/W architecture or structure, which consists of fatty phases dispersed in an aqueous phase, has an external aqueous phase, and therefore if the composition according to the present invention has the O/W architecture or structure, it can provide a pleasant feeling during use because of the feeling of immediate freshness that the aqueous phase can provide.

### [Process and Use]

It is preferable that the composition according to the present invention be a cosmetic or dermatological composition, preferably a cosmetic composition, and more preferably a cosmetic composition for a keratin substance such as skin.

The composition according to the present invention can be used for a non-therapeutic process, such as a cosmetic process, for treating a keratin substance such as skin, mucous membranes, and/or scalp, by being applied to the keratin substance.

Thus, the present invention also relates to a cosmetic process for treating a keratin substance such as skin, comprising the step of applying the composition according to the present invention to the keratin substance.

The present invention may also relate to a use of the composition according to the present invention as a cosmetic product or in a cosmetic product such as care products and make-up products, for body and/or facial skin and/or mucous membranes and/or the scalp.

In other words, the composition according to the present invention can be used, as it is, as a cosmetic product. Alternatively, the composition according to the present invention can be used as an element of a cosmetic product. For example, the composition according to the present invention can be added to or combined with any other elements to form a cosmetic product.

The care product may be a lotion, in particular a milky lotion, a cream, and the like. The make-up product may be a foundation, a lipstick, a lip gloss, an eye shadow, and the like.

Another aspect of the present invention relates to a use of:
(a) at least one amino acid surfactant, preferably at least one amino acid gemini surfactant;
(b) at least one polyglyceryl fatty acid ester; and
(d) at least one fatty alcohol,
wherein
the HLB value of the (b) polyglyceryl fatty acid ester(s) is 6 or more, preferably 7 or more, and more preferably 8 or more, and
the fatty acid moiety of the (b) polyglyceryl fatty acid ester comprises carbon atoms of 16 or less, preferably 14 or less, and more preferably 12 or less,
in a composition comprising
(c) at least one UV filter, preferably at least one organic UV filter, in order to make the composition stable and/or make the composition be capable of forming a homogeneous film.

The above explanations regarding ingredients (a) to (d), as well as the optional ingredients, for the composition according to the present invention can apply to those for the above use according to the present invention. The explanations regarding the preparation and forms of the composition according to the present invention can also apply to those of the composition described in the above use.

### EXAMPLES

The present invention will be described in more detail by way of examples which however should not be construed as limiting the scope of the present invention.

### Examples 1-10 and Comparative Examples 1-10

The following compositions according to Examples 1-10 and Comparative Examples 1-10, shown in Tables 1 and 2, were prepared by mixing the ingredients shown in Tables 1 and 2 as follows:
(1) mixing the ingredients of Phase A at 60-65° C. to form a uniform mixture of Phase A;
(2) adding the ingredients of Phase B to the uniform mixture of Phase A at 60-65° C. followed by mixing with a homogenizer at 60-65° C. to form a uniform mixture of Phases A and B;
(3) adding the ingredients of Phase C to the uniform mixture of Phases A and B followed by mixing by a homogenizer at 60-65° C. to form an aqueous phase;
(4) separately mixing the ingredients of Phase D at 75-80° C. to form an oil phase;
(5) mixing the aqueous and oil phases at 60-65° C. with a homogenizer to obtain a uniform mixture;
(6) cooling the uniform mixture of Phases A, B, C and D to about 40° C.; and
(7) adding the ingredient of Phase E to the uniform mixture of Phases A, B, C and D at about 40° C. followed by mixing with a homogenizer.

The compositions according to Examples 1-10 and Comparative Examples 1-10 were in the form of an O/W emulsion.

The numerical values for the amounts of the components shown in Tables 1 and 2 are all based on by weight” as active ingredients.

TABLE 1

| | Ingredients | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|---|
| A | Water | qsp 100 | qsp 100 | qsp 100 | qsp 100 | qsp 100 | qsp 100 | qsp 100 | qsp 100 | qsp 100 | qsp 100 |
| | Caprylyl Glycol | 0.3 | 0.3 | 0.3 | 0.3 | 0.3 | 0.3 | 0.3 | 0.3 | 0.3 | 0.3 |
| | Glycerin | 3 | 3 | 3 | 3 | 3 | 3 | 3 | 3 | 3 | 3 |
| | Butylene Glycol | 2.5 | 2.5 | 2.5 | 2.5 | 2.5 | 2.5 | 2.5 | 2.5 | 2.5 | 2.5 |
| | Trisodium Ethylenediamine | 0.2 | 0.2 | 0.2 | 0.2 | 0.2 | 0.2 | 0.2 | 0.2 | 0.2 | 0.2 |
| | Disuccinate | | | | | | | | | | |
| | Phenoxyethanol | 0.6 | 0.6 | 0.6 | 0.6 | 0.6 | 0.6 | 0.6 | 0.6 | 0.6 | 0.6 |
| | Hydroxyacetophenone | 0.2 | 0.2 | 0.2 | 0.2 | 0.2 | 0.2 | 0.2 | 0.2 | 0.2 | 0.2 |
| | Potassium Hydroxide | 0.3 | 0.3 | 0.3 | 0.3 | 0.3 | 0.3 | 0.3 | 0.3 | 0.3 | 0.3 |
| B | Xanthan Gum | 0.3 | 0.3 | 0.3 | 0.3 | 0.3 | 0.3 | 0.3 | 0.3 | 0.3 | 0.3 |
| | Ammonium | 0.4 | 0.4 | 0.4 | 0.4 | 0.4 | 0.4 | 0.4 | 0.4 | 0.4 | 0.4 |
| | Acryloyldimethyltaurate/VP | | | | | | | | | | |
| | Copolymer | | | | | | | | | | |
| | Carbomer | 0.15 | 0.15 | 0.15 | 0.15 | 0.15 | 0.15 | 0.15 | 0.15 | 0.15 | 0.15 |
| C | Sodium Dilauramidoglutamide | 0.2 | 0.2 | 0.2 | 0.2 | 0.2 | 0.2 | 0.2 | 0.2 | 0.2 | 0.2 |
| | Lysine | | | | | | | | | | |
| | Polyglyceryl-6 Caprylate (HLB: 15) | 1 | — | — | — | — | — | — | — | — | — |
| | Polyglyceryl-4 Caprate (HLB: 14) | — | 1 | 1 | 1 | 1 | — | — | — | — | 1 |
| | Polyglyceryl-6 Dicaprate (HLB: 10) | — | — | — | — | — | 1 | — | — | — | — |
| | Polyglyceryl-2 Laurate (HLB: 8.5) | — | — | — | — | — | — | 1 | — | — | — |
| | Polyglyceryl-5 Laurate (HLB: 11) | — | — | — | — | — | — | — | 1 | — | — |
| | Polyglyceryl-10 Laurate (HLB: 16) | — | — | — | — | — | — | — | — | 1 | — |
| D | Cetyl Alcohol | — | 1 | 0.5 | — | — | — | — | — | — | — |
| | Arachidyl Alcohol (and) Behenyl | 0.5 | — | — | 1 | 0.5 | 0.5 | 0.5 | 0.5 | 0.5 | 0.5 |
| | Alcohol | | | | | | | | | | |
| D | Butyl Methoxydimenzoylmethane | 2 | 2 | 2 | 2 | 2 | 2 | 2 | 2 | 2 | 3 |
| | Bis-Ethylhexyloxyphenol | 3 | 3 | 3 | 3 | 3 | 3 | 3 | 3 | 3 | — |
| | Methoxyphenyl Triazine | | | | | | | | | | |
| | Ethylhexyl Triazone | 4 | 4 | 4 | 4 | 4 | 4 | 4 | 4 | 4 | — |
| | Drometrizole Trisiloxane | 2 | 2 | 2 | 2 | 2 | 2 | 2 | 2 | 2 | — |
| | Ethylhexyl Salicylate | — | — | — | — | — | — | — | — | — | 5 |
| | Octocrylene | — | — | — | — | — | — | — | — | — | 7 |
| | Homosalate | — | — | — | — | — | — | — | — | — | 10 |
| | C12-15 Alkyl Benzoate | 4.5 | 4.5 | 4.5 | 4.5 | 4.5 | 4.5 | 4.5 | 4.5 | 4.5 | — |
| | Isopropyl Lauroyl Sarcosinate | 3 | 3 | 3 | 3 | 3 | 3 | 3 | 3 | 3 | — |
| | Diisopropyl Sebacate | 3 | 3 | 3 | 3 | 3 | 3 | 3 | 3 | 3 | — |
| | Shea Butter | 1 | 1 | 1 | 1 | 1 | 1 | 1 | 1 | 1 | 1 |
| | C15-19 Alkane | 4 | 4 | 4 | 4 | 4 | 4 | 4 | 4 | 4 | 4 |
| | Pentaerythrityl Tetra-di-t-butyl | 0.5 | 0.5 | 0.5 | 0.5 | 0.5 | 0.5 | 0.5 | 0.5 | 0.5 | 0.5 |
| | Hydroxyhydrocinnamate | | | | | | | | | | |
| | Poly C10-30 Alkyl Acrylate | 0.5 | 0.5 | 0.5 | 0.5 | 0.5 | 0.5 | 0.5 | 0.5 | 0.5 | 0.5 |
| E | Mica | 1 | 1 | 1 | 1 | 1 | 1 | 1 | 1 | 1 | 1 |
| | Titanium Dioxide | 3 | 3 | 3 | 3 | 3 | 3 | 3 | 3 | 3 | 3 |
| | Silica | 1 | 1 | 1 | 1 | 1 | 1 | 1 | 1 | 1 | 1 |

| | | | | | | | | | | |
|---|---|---|---|---|---|---|---|---|---|---|
| Film Homogeneity | 5 | 4.5 | 5 | 5 | 5 | 5 | 5 | 4 | 4 | 5 |
| Stability | Good | Good | Good | Good | Good | Good | Good | Good | Good | Good |

TABLE 2

| | | Comp. | Comp. | Comp. | Comp. | Comp. | Comp. | Comp. | Comp. | Comp. | Comp. |
|---|---|---|---|---|---|---|---|---|---|---|---|
| | Ingredients | Ex. 1 | Ex. 2 | Ex. 3 | Ex. 4 | Ex. 5 | Ex. 6 | Ex. 7 | Ex. 8 | Ex. 9 | Ex. 10 |
| A | Water | qsp 100 | qsp 100 | qsp 100 | qsp 100 | qsp 100 | qsp 100 | qsp 100 | qsp 100 | qsp 100 | qsp 100 |
| | Caprylyl Glycol | 0.3 | 0.3 | 0.3 | 0.3 | 0.3 | 0.3 | 0.3 | 0.3 | 0.3 | 0.3 |
| | Glycerin | 3 | 3 | 3 | 3 | 3 | 3 | 3 | 3 | 3 | 3 |
| | Butylene Glycol | 2.5 | 2.5 | 2.5 | 2.5 | 2.5 | 2.5 | 2.5 | 2.5 | 2.5 | 2.5 |
| | Trisodium Ethylenediamine | 0.2 | 0.2 | 0.2 | 0.2 | 0.2 | 0.2 | 0.2 | 0.2 | 0.2 | 0.2 |
| | Disuccinate | | | | | | | | | | |
| | Phenoxyethanol | 0.6 | 0.6 | 0.6 | 0.6 | 0.6 | 0.6 | 0.6 | 0.6 | 0.6 | 0.6 |
| | Hydroxyacetophenone | 0.2 | 0.2 | 0.2 | 0.2 | 0.2 | 0.2 | 0.2 | 0.2 | 0.2 | 0.2 |
| | Potassium Hydroxide | 0.3 | 0.3 | 0.3 | 0.3 | 0.3 | 0.3 | 0.3 | 0.3 | 0.3 | 0.3 |
| B | Xanthan Gum | 0.3 | 0.3 | 0.3 | 0.3 | 0.3 | 0.3 | 0.3 | 0.3 | 0.3 | 0.3 |
| | Ammonium | 0.4 | 0.4 | 0.4 | 0.4 | 0.4 | 0.4 | 0.4 | 0.4 | 0.4 | 0.4 |
| | Acryloyldimethyltaurate/VP | | | | | | | | | | |
| | Copolymer | | | | | | | | | | |
| | Carbomer | 0.15 | 0.15 | 0.15 | 0.15 | 0.15 | 0.15 | 0.15 | 0.15 | 0.15 | 0.15 |
| C | Sodium Dilauramidoglutamide | 0.2 | 0.2 | 0.2 | 0.2 | 0.2 | — | — | 0.2 | — | 0.2 |
| | Lysine | | | | | | | | | | |
| | Sodium Lauryl Sulfate | — | — | — | — | — | 0.2 | — | — | — | — |
| | Stearic Acid | — | — | — | — | — | — | 0.2 | — | — | — |
| | Sorbitan Oleate | — | — | — | — | — | — | — | — | — | 0.5 |
| | Polyglyceryl-10 Laurate (HLB: 16) | — | — | — | — | 1 | 1 | 1 | — | — | — |
| | Polyglyceryl-10 Dioleate (HLB: 12) | 1 | — | — | — | — | — | — | — | — | — |
| | Polyglyceryl-4 Isostearate (HLB: 5) | — | 1 | — | — | — | — | — | — | — | — |
| | Glyceryl Isostearate (HLB: 4) | — | — | 1 | — | — | — | — | — | — | — |
| D | Arachidyl Alcohol (and) Behenyl | 0.5 | 0.5 | 0.5 | 0.5 | — | 0.5 | 0.5 | — | — | — |
| | Alcohol | | | | | | | | | | |
| D | Butyl Methoxydimenzoylmethane | 2 | 2 | 2 | 2 | 2 | 2 | 2 | 2 | 2 | 3 |
| | Bis-Ethylhexyloxyphenol | 3 | 3 | 3 | 3 | 3 | 3 | 3 | 3 | 3 | — |
| | Methoxyphenyl Triazine | | | | | | | | | | |
| | Ethylhexyl Triazone | 4 | 4 | 4 | 4 | 4 | 4 | 4 | 4 | 4 | — |
| | Drometrizole Trisiloxane | 2 | 2 | 2 | 2 | 2 | 2 | 2 | 2 | 2 | — |
| | Ethylhexyl Salicylate | — | — | — | — | — | — | — | — | — | 5 |
| | Octocrylene | — | — | — | — | — | — | — | — | — | 7 |
| | Homosalate | — | — | — | — | — | — | — | — | — | 10 |
| | C12-15 Alkyl Benzoate | 4.5 | 4.5 | 4.5 | 4.5 | 4.5 | 4.5 | 4.5 | 4.5 | 4.5 | — |
| | Isopropyl Lauroyl Sarcosinate | 3 | 3 | 3 | 3 | 3 | 3 | 3 | 3 | 3 | — |
| | Diisopropyl Sebacate | 3 | 3 | 3 | 3 | 3 | 3 | 3 | 3 | 3 | — |
| | Shea Butter | 1 | 1 | 1 | 1 | 1 | 1 | 1 | 1 | 1 | 1 |
| | C15-19 Alkane | 4 | 4 | 4 | 4 | 4 | 4 | 4 | 4 | 4 | 4 |
| | Pentaerythrityl Tetra-di-t-butyl | 0.5 | 0.5 | 0.5 | 0.5 | 0.5 | 0.5 | 0.5 | 0.5 | 0.5 | 0.5 |
| | Hydroxyhydrocinnamate | | | | | | | | | | |
| | Poly C10-30 Alkyl Acrylate | 0.5 | 0.5 | 0.5 | 0.5 | 0.5 | 0.5 | 0.5 | 0.5 | 0.5 | 0.5 |
| E | Mica | 1 | 1 | 1 | 1 | 1 | 1 | 1 | 1 | 1 | 1 |
| | Titanium Dioxide | 3 | 3 | 3 | 3 | 3 | 3 | 3 | 3 | 3 | 3 |
| | Silica | 1 | 1 | 1 | 1 | 1 | 1 | 1 | 1 | 1 | 1 |

| | | | | | | | | | | |
|---|---|---|---|---|---|---|---|---|---|---|
| Film Homogeneity | 2 | 2.5 | 2 | 1 | 2 | 2 | 2.5 | 1 | 1 | 2 |
| Stability | Poor | Poor | Poor | Good | Poor | Poor | Poor | Poor | Poor | Poor |

### [Evaluations]

### (Film Homogeneity)

Each composition in the same amount was applied on a polypropylene sheet (0.2 mm PX-P from Sekisui Kagaku Co. Ltd) by an applicator called “Elecometer 4340” (Elecometer K.K.) with a weight of 1 kg, and dried to form a film on the sheet. Then, the applied sheet was exposed to UV (wavelength 365 nm) by using a UV lamp (UV black ray B-100AP).

The homogeneity of the film on the sheet was evaluated with visual observation. A film with good film homogeneity showed homogeneous intensive black color. On the other hand, a film with poor homogeneity showed non-homogeneous translucent blue color. The picture of each film was taken and scored from 1 to 5. (1 means low film homogeneity while 5 means high film homogeneity).

The results of the above evaluation as an average score are shown in Table 1.

### (Stability)

3 g of each composition was put in a 30 ml beaker, and stirred with a small stirrer bar (0.76 g, 1.5 cm length) at a shear speed of 400 rpm at room temperature for 5 minutes. Then, the composition was observed under microscopy (Olympus Polarizing Microscope BX51-P) to evaluate the stability of the composition in accordance with the following criteria.
Good: No phase separation or no aggregation
Poor: Phase separation or aggregation

The results of the above evaluation are shown in Table 1.

### Example 11

The following composition according to Example 11, shown in Table 3, was prepared by mixing the ingredients shown in Table 3 as follows:
(1) mixing the ingredients of Phase A at 60-65° C. to form a uniform mixture of Phase A; (2) adding the ingredients of Phase B to the uniform mixture of Phase A at 60-65° C. followed by mixing with a homogenizer at 60-65° C. to form a uniform mixture of Phases A and B;
(3) adding the ingredients of Phase C to the uniform mixture of Phases A and B followed by mixing by a homogenizer at 60-65° C. to form an aqueous phase;
(4) separately mixing the ingredients of Phase D at 75-80° C. to form an oil phase; (5) mixing the aqueous and oil phases at 60-65° C. with a homogenizer to obtain a uniform mixture;
(6) cooling the uniform mixture of Phases A, B, C and D to about 40° C.; and
(7) adding the ingredient of Phase E to the uniform mixture of Phases A, B, C and D at about 40° C. followed by mixing with a homogenizer.

The composition according to Example 11 was in the form of an O/W emulsion.

The numerical values for the amounts of the components shown in Table 3 are all based on “% by weight” as active ingredients.

TABLE 3

| | Ingredients | Ex. 11 |
|---|---|---|
| A | Water | qsp 100 |
| | Caprylyl Glycol | 0.3 |
| | Glycerin | 3 |
| | Butylene Glycol | 2.5 |
| | Trisodium Ethylenediamine Disuccinate | 0.2 |
| | Phenoxyethanol | 0.6 |
| | Hydroxyacetophenone | 0.2 |
| | Potassium Hydroxide | 0.3 |
| B | Xanthan Gum | 0.3 |
| | Ammonium Acryloyldimethyltaurate/VP Copolymer | 0.4 |
| | Carbomer | 0.15 |
| C | Sodium Dilauramidoglutamide Lysine | 0.2 |
| | Polyglyceryl-4 Caprate (HLB:14) | 1 |
| D | Arachidyl Alcohol (and) Behenyl Alcohol | 0.5 |
| | Butyl Methoxydimenzoylmethane | 2 |
| | Bis-Ethylhexyloxyphenol Methoxyphenyl Triazine | 3 |
| | Ethylhexyl Triazone | 4 |
| | Drometrizole Trisiloxane | 2 |
| | C12-15 Alkyl Benzoate | 4.5 |
| | Isopropyl Lauroyl Sarcosinate | 3 |
| | Diisopropyl Sebacate | 3 |
| | Shea Butter | 1 |
| | C15-19 Alkane | 4 |
| | Pentaerythrityl Tetra-di-t-butyl | 0.5 |
| | Hydroxyhydrocinnamate | |
| | Poly C10-30 Alkyl Acrylate | 0.5 |
| E | Mica | 0.5 |
| | Titanium Dioxide | 1.5 |
| | Silica | 2 |

| | |
|---|---|
| SPF in vivo | 43 |
| Texture | Good |

### [Evaluations]

### (SPF In Vivo)

The Sun Protection Factor (SPF) in vivo of the composition according to Example 11 was determined in accordance with ISO/EN 24444 (2019) Cosmetics-Sun protection test methods—In vivo determination of the sun protection factor (SPF). This test was performed using a UV simulator (UV source: Xenon lamp: Solar Light type Multiport 601-300W, v2.5 Filter WG320, Spectrum: 290 to 400 nm).

The results are shown in Table 3.

### (Texture)

The texture during application of the composition according to Example 11 to the skin was evaluated by applying the composition to a forearm of a panelist at an amount of 2 mg/cm² and then assessing the friction force felt between the fingers and the surface of the forearm by the panelist. The texture was evaluated in accordance with the following criteria.
Good: Smooth or not sticky
Poor: Not smooth or sticky

The results are shown in Table 3.

### (Summary)

As shown in Table 1, the compositions according to Examples 1-10 are stable and can form a homogeneous film.

As shown in Table 3, the composition according to Example 11 can provide good UV protection and good texture of smooth or non-sticky feeling to touch during application. The composition according to Example 11 is very similar to the composition according to Example 5. Thus, it is clear that the compositions according to Examples 1-10 can also provide good UV protection and good texture of smooth or non-sticky feeling to touch during application.

On the other hand, Table 2 shows that absence of any of the ingredients (a) to (d) in the composition according to the present invention negatively affects the stability of the composition and the ability of forming a homogeneous film by the composition. It is clear that the formation of a non-homogeneous film would deteriorate UV protection.

For example, Comparative Examples 1-3 demonstrate that the compositions including a polyglyceryl fatty acid ester with a fatty acid moiety including more than 16 carbon atoms are not stable and cannot form a homogeneous film.

Comparative Example 4 demonstrates that a composition including no polyglyceryl fatty acid ester cannot form a homogeneous film.

Comparative Example 5 demonstrates that a composition including no fatty alcohol is not stable and cannot form a homogeneous film.

Comparative Examples 6 and 7 demonstrate that a composition including no amino acid surfactant is not stable and cannot form a homogeneous film, even if the composition includes another anionic surfactant (sodium lauryl sulfate or stearic acid with potassium hydroxide) other than the amino acid surfactant.

Comparative Example 8 demonstrates that a composition including neither polyglyceryl fatty acid ester nor fatty alcohol is not stable and cannot form a homogeneous film.

Comparative Example 9 demonstrates that a composition including neither polyglyceryl fatty acid ester, amino acid surfactant, nor fatty alcohol is not stable and cannot form a homogeneous film.

Comparative Example 10 demonstrates that a composition including neither polyglyceryl fatty acid ester nor fatty alcohol is not stable and cannot form a homogeneous film, even if the composition includes another nonionic surfactant (sorbitan oleate) other than the polyglyceryl fatty acid ester.

1. A composition comprising:
(a) at least one amino acid surfactant;
(b) at least one polyglyceryl fatty acid ester;
(c) at least one UV filter; and
(d) at least one fatty alcohol,
wherein
the HLB value of the (b) at least one polyglyceryl fatty acid ester is 6 or more, and
the fatty acid moiety of the (b) at least one polyglyceryl fatty acid ester comprises carbon atoms of 16 or less.

2. The composition according to claim 1, wherein the (a) at least one amino acid surfactant is selected from the compounds represented by the formula (A): 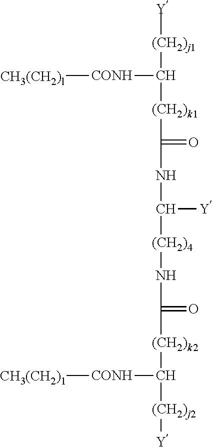
wherein
Y′ independently denotes a carboxylic acid group or an alkaline salt of a carboxylic acid group,
j1, k1, j2 and k2 denote integers such that (j1, k1, j2, k2)=any of (2, 0, 2, 0), (2, 0, 0, 2), (0, 2, 2, 0) and (0, 2, 0, 2), and
l denotes an integer from 6 to 16.

3. The composition according to claim 1, wherein the (a) at least one amino acid surfactant is selected from the group consisting of sodium dilauramidoglutamide lysine, sodium dimyristoylglutamide lysine, and sodium distearoylglutamide lysine, and a mixture thereof.

4. The composition according to claim 1, wherein the amount of the (a) at least one amino acid surfactant in the composition ranges from 0.01% to 10% by weight relative to the total weight of the composition.

5. The composition according to claim 1, wherein the (b) at least one polyglyceryl fatty acid ester is selected from the group consisting of PG2 laurate, PG4 caprate, PG5 laurate, PG6 dicaprate, PG6 caprylate, PG10 laurate, and a mixture thereof.

6. The composition according to claim 1, wherein the amount of the (b) at least one polyglyceryl fatty acid ester in the composition ranges from 0.01% to 10% by weight relative to the total weight of the composition.

7. The composition according to claim 1, wherein the (c) at least one UV filter is selected from organic UV filters.

8. The composition according to claim 1, wherein the amount of the (c) at least one UV filter in the composition ranges from 1% to 30% by weight relative to the total weight of the composition.

9. The composition according to claim 1, wherein the (d) at least one fatty alcohol is selected from the group consisting of cetyl alcohol, stearyl alcohol, arachidyl alcohol, behenyl alcohol, and a mixture thereof.

10. The composition according to claim 1, wherein the amount of the (d) at least one fatty alcohol in the composition ranges from 0.01% to 10% by weight relative to the total weight of the composition.

11. The composition according to claim 1, wherein the composition further comprises (e) water.

12. The composition according to claim 11, wherein the amount of the (e) water in the composition ranges from 40% to 70% by weight relative to the total weight of the composition.

13. The composition according to claim 11, wherein the composition is in the form of an O/W emulsion.

14. The composition according to claim 1, wherein the composition is a cosmetic composition.

15. A cosmetic process for treating a keratin substance, comprising:
applying the composition according to claim 1 to the keratin substance.
